(19) United States
(12) Patent Application Publication
(10) Pub. No.: US 2011/0202070 A1
(43) Pub. Date: Aug. 18, 2011

(54) ENDOLUMINAL ROBOTIC SYSTEM

(76) Inventors: Paolo Dario, Livorno (IT); Alfred Cuschieri, Lucca (IT); Arianna Menciassi, Pontedera (IT); Pietro Valdastri, Livorno (IT); Kanako Harada, Syunan-shi (Yamaguchi) (JP)

(21) Appl. No.: 13/124,651

(22) PCT Filed: Oct. 16, 2009
(86) PCT No.: PCT/IB09/54561

§ 371 Date: Apr. 15, 2011

(51) Int. Cl. A61B 19/00

(52) U.S. Cl. 606/130

(57) ABSTRACT

A micro-robotic platform and a method for deploying the platform in a body cavity for performing endoluminal surgical interventions in a fully bimanual fashion. The platform comprises a first (1) and a second (2) surgical robot each provided with a surgical tool (5, 6) and being configured to be attached to the body cavity wall. The first and the second surgical robot each comprises a first (11) and a second (12) snake-like robotic unit, the first snake-like robotic unit (11) comprising a first central unit (13) and first articulated attachment means (7a, b; 8a, b; 9) extending from said first central unit (13) for attaching the first central unit to the body cavity wall. The second snake-like robotic unit (12) comprises a second central unit (14), second articulated attachment means (7c; 8c; 9) for attaching the second central unit to the body cavity wall and an articulated arm (3, 4) bearing the surgical tool (5, 6), the second articulated attachment means and the articulated arm extending from the second central unit. Releasable connection means (15) are provided on the first and second central units (13, 14) to connect the first central unit to the second central unit releasably to form each of the first and second surgical robot within the body cavity deployed in such a way to allow a surgical procedure to be performed in a true bimanual way.

### FIELD OF THE INVENTION

The present invention generally relates to the field of endoluminal surgery and more particularly relates to an endoluminal bimanual micro-robotic platform and a method for deploying the micro-robotic platform in a body cavity such as the gastric cavity.

### BACKGROUND OF THE INVENTION

In traditional open surgery, physical and rigid links exist between the surgeon and the patient's organs. The instruments are hand-held and operated under direct binocular vision.

With the introduction of laparoscopic techniques, the direct physical links between the surgeon and the patient's organs are represented by the trocars, which are used for the insertion of different instruments, energised dissection devices and staplers, all having a remote end-effector and proximal actuation i.e., the surgeon's hand.

Surgical tele-operated robots, such as the Da Vinci® system, are considered an important on-going evolution in minimally invasive surgery because, whilst the main features of surgical execution are retained, the actuation of bimanual tools is remote from the patient and is performed by the surgeon operating from a console. The Da Vinci® wrist is remotely driven by actuators at the proximal end of the tool module through cable drives.

Recent examples of autonomous surgical robots include the inchworm-type devices. Self-propelled robotic endoscopes have been developed for navigation in tubular organs [K. Ikuta et al., Hyper-redundant active endoscope for minimum invasive surgery, Proc. First Int. Symp. on Medical Robotics and Computer Assisted Surgery, Pittsburg, Pa., 1994; A. B. Slatkin et al., The development of a robotic endoscope, Intelligent Robots and Systems 95. ‘Human Robot Interaction and Cooperative Robots’, Proceedings. 1995 IEEE/RSJ International Conference, vol. 2, pp. 162-171, 5-9 Aug. 1995; A. Menciassi et al., Robotic solutions and mechanisms for a semi-autonomous endoscope, Intelligent Robots and System, 2002. IEEE/RSJ International Conference, vol. 2, pp. 1379-1384, 2002].

Hyper-redundant robotic structures, essentially snake-like structures, improve manipulation performance in complex and highly constrained environments. They have been used in several fields of industry as well as bimanual haptic interfaces and have been also proposed for design of endoscopic robots. Existing hyper-redundant robotic structures, also for surgery, are generally cable actuated from external driving systems and thus are mechanically connected with the outside world.

In flexible interventional endoscopy, the rigid link between the surgeon and the organs becomes progressively weaker as the mechanical constraints are transferred from outside the body (e.g. the hand held device, the instruments inside the trocar, etc.) to lumen of an internal hollow organ. Mechanically, as exemplified by the autonomous colonoscopes, the rigid transmission from outside is removed. A surgical robotic system for flexible endoscopy is disclosed in WO2007/111571. A pair of robotic arms ending with surgical tools extend from the distal end of a flexible endoscope. US2005/096502 discloses a surgical device for use in laparoscopy or surgical endoscopy comprising an elongated body with a plurality of arms carrying surgical tools extending from the distal end.

The current robotic solutions for gastric surgical procedures are based on bulky robotic units and still require several incisions in the patient's abdomen. On the other hand, current endoluminal procedures for gastrointestinal tract surgery (a subset of NOTES—Natural Orifice Transluminal Endoscopic Surgery—procedures) are still not effective, since they are usually performed by a single flexible instrument, having external cable actuation. Furthermore, this approach has not yet exploited the benefits of robotics. Significant advance is expected with the integration of computer-assisted surgery, flexible endoscopy and tele-operated laparoscopy to access the abdominal cavity through natural orifices

Micro-robots introduced into the peritoneal cavity in pigs through a standard 12 mm laparoscopic trocar after gas insufflation have been reported recently as an adjunct to laparoscopic surgery [D. Oleynikov et al., Miniature robots can assist in laparoscopic cholecystectomy, Surg Endosc., vol. 19, pp. 473-476, DOI: 10.1007/s00464-004-8918-6, 2005]. On this subject see also WO2007/149559, relating to a robotic surgical device insertable in the patient's body and positionable within the patient's body using an external magnet. A pair of arms extend from a body housing magnets interacting with an outer magnetic handle for controlling the positioning of the device. Each arm ends with a surgical tool and is connected to the body through a shoulder joint with two degrees of freedom. Each arm comprises two arm portions connected to one another through an elbow joint with one degree of freedom.

The robotic device according to WO2007/149559, as well those proposed for flexible endoscopy cited above, is unable to perform true bimanual operation due to the fact that the arms bearing the surgical tool extend from a common origin and, despite the degrees of freedom of the arms, their possibility of co-operation is limited by dimensional and spatial factors and the workspace results quite limited. Moreover, the single arms extend from a point which is fixed to the abdominal cavity by magnetic means. All the forces exerted by the arms must be supported by the same force which maintains the common origin in contact with the abdominal wall. This limits the range of force that can be exerted by the tools. In addition to that, a threshold between the working space (proportional to the number of links, thus to the length, of each arm) and the maximum force (limited by the maximum torque that the magnetic link can support, thus inversely proportional to the arm length) must be fixed.

Furthermore, existing research has shown that there is a pressing need for developing a novel surgical approach based on micro-robotics instead of attempting instrumental technologies for the existing operating flexible endoscopes. The multi-capsule robotic device disclosed by Menciassi et al., Biomedical Robotics and Biomechatronics, 2008, BIOROB 2008, 2nd IEEE RAS&EMBS INT. CONF. ON, IEEE, Piscataway, N.J., USA, 238-243, Oct. 19, 2008, follows this trend, but, like the device disclosed in WO2007/149559, cannot work in a bimanual way and hence is affected by the same drawbacks. Moreover, a local self-assembling technique of the device from the single component capsules swallowed by the patient is foreseen which is very difficult to carry out with the required precision and velocity.

### SUMMARY OF THE INVENTION

The object of the present invention is to provide a micro-robotic platform for advanced endoluminal surgery insertable for deployment in a body cavity to perform surgical procedures with a true bimanual robotic operation.

A particular object of the present invention is to provide an endoluminal micro-robotic platform of the above mentioned type formed by at least two deployable surgical robots capable of being coupled in a body cavity.

A further object of the present invention is to provide an endoluminal micro-robotic platform of the above mentioned type in which the surgical robots have an higher number of degrees of freedom and an increased stability, are able to withstand to higher forces and have better manipulation performances than the prior art similar devices.

It is still another object of the present invention to provide a method for deploying the endoluminal micro-robotic platform of the above mentioned type in a body cavity such as the gastric cavity.

These objects are achieved with the endoluminal micro-robotic platform and the method for its deployment in a body cavity according to the invention, the main features of which are stated in the independent claims 1 and 10. Further important features are set forth in the dependent claims.

### BRIEF DESCRIPTION OF THE DRAWINGS

The features and the advantages of the endoluminal micro-robotic platform and the method for its deployment in a body cavity according to the invention will be apparent from the following description of an embodiment thereof given by way of a non-limiting example with reference to the attached drawings, in which

FIG. 1 is a schematic overall view of the endoluminal micro-robotic platform according to the invention operatively deployed in a body cavity;

FIG. 2 is a perspective view of an assembled deployable surgical robot;

FIG. 3 is a perspective view of a snake-like robotic unit;

FIG. 4 is a perspective view of an arm of the robotic unit of FIG. 3;

FIG. 5 is a longitudinal view of the arm of FIG. 4 in an extended condition;

FIG. 6 is another longitudinal view of the arm of FIG. 4, axially rotated by 90° relative to FIG. 5;

FIG. 7 is a cross sectional view of the arm of the robotic unit taken along lines VII-VII of FIG. 6;

FIG. 8 is a longitudinal sectional view of the arm of the robotic unit taken along lines VIII-VIII of FIG. 6;

FIGS. 9 to 19 illustrate the steps of the method for deploying the robotic platform in a body cavity such as the stomach.

### DETAILED DESCRIPTION OF THE INVENTION

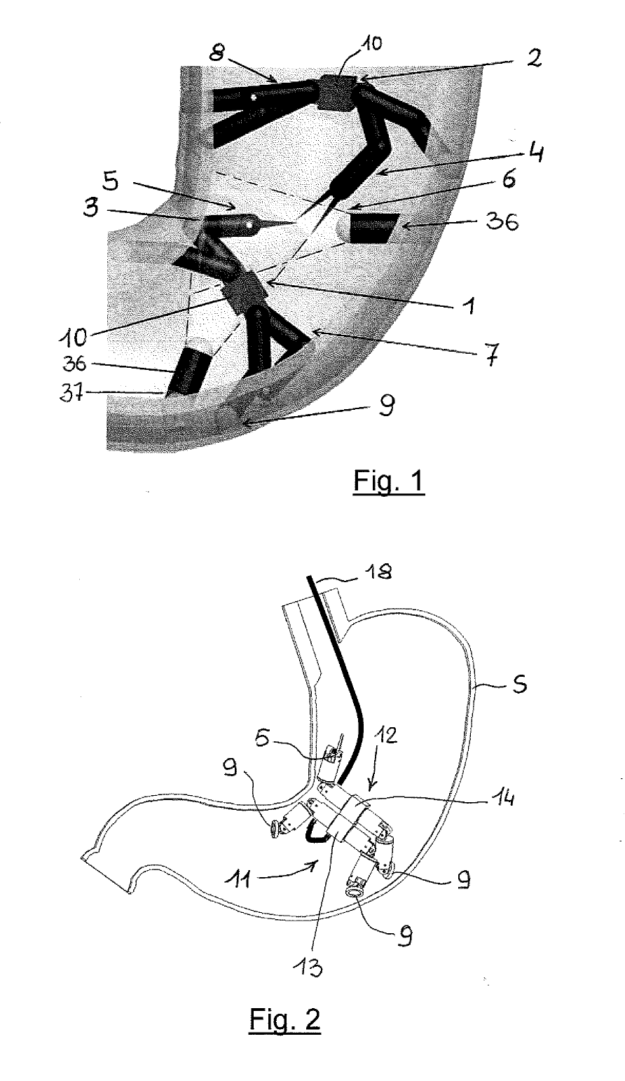

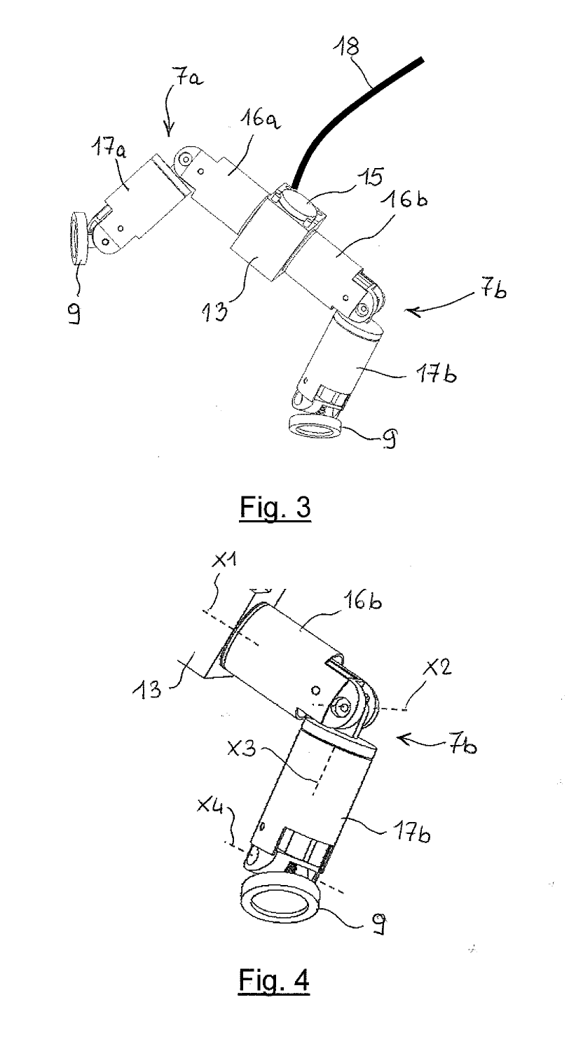

As used in the present specification the term “robotic platform” is meant as a robotic framework or a set of robotic components when assembled and deployed in a body cavity to perform a surgical procedure. Likewise, the term “bimanual” is meant as having two surgical tools capable of being operated in the same way as the hands of the surgeon, with substantially the same degrees of freedom. A “snake-like robot” is defined as a flexible or articulated robotic functional unit, as in FIG. 3, which can be inserted into human body cavities through natural orifices thanks to its cylindrical shape. A single “snake-like robot” may connect with one or more similar “snake-like robots” to form a surgical robot, as shown in FIG. 2.

With reference to FIGS. 1 to 4, the endoluminal micro-robotic platform according to the invention comprises a first and a second deployable surgical robot, generally indicated as 1 and 2, each being equipped with an operating arm 3 and 4, bearing a surgical tool 5 and 6 (either an operative tool or an assistive tool or a monitoring sensor) at the respective free ends, and with three attachment legs 7a, b, c, and 8a, b, c respectively configured to permit a stable hold of the surgical robots to the wall of a body cavity, for example the stomach S, as shown in the figures. In particular, the attachment legs 7a, b, c and 8a, b, c are equipped with an attachment device 9 at their free ends, through which they are connected to the wall of the body cavity. By way of example, the attachment device 9 may be a mucho-adhesive polymer layer or an air suction device or a magnetic device in combination with external or internal magnets. The operative arms 3 and 4 and the attachment legs 7a, b, c and 8a, b, c of each surgical robot, extend from a respective body 10 connected to a cable 18 for energy supply and data transmission that passes through an inserting port to be connected to an external unit placed out of the patient's body.

As shown in FIG. 2, each surgical robot 1 and 2 is formed by a first and a second snake-like robotic unit 11, 12. The first snake-like robotic unit comprises a central unit 13 and two attachment legs 7a, b (or 8a, b) extending therefrom. The second snake-like robotic unit 12 comprises a central unit 14, the attachment leg 7c (8c) and the operating arm 3 (4), both extending from the central unit 13 (14). The central units 13 and 14 embed a local electronic control circuitry and an electro-mechanical connector 15 to connect reversibly the central unit 13 of the first snake-like robotic unit 11 to the central unit 14 of the second snake-like robotic unit 12 to form the body 10 of the surgical robot 1 (2). The connection surface can have connectors, for energy or data transmission between the central units 13 and 14.

Each attachment leg 7a, b, c and 8a, b, c and each operating arm 3 and 4 has four degrees of freedom. In particular, as shown in detail in FIGS. 3 and 4, each attachment leg 7a and 7b (the same applies to attachment legs 7c and 8a, b, c and to operating arms 3 and 4)) is formed by a proximal leg portion 16a and 16b respectively extending from opposite parts of the central unit 13 and a distal leg portion 17a and 17b respectively extending from the free ends of the proximal leg portions 16a and 16b. An attachment device 9 is placed at the free ends of each of the distal leg portions 16b, 17b. The proximal leg portion 16a (16b) is connected for axial rotation about its longitudinal axis X1 to the central unit 13. The distal leg portion 17a (17b) is connected for bending about a transverse axis X2 to the proximal leg portion 16a (16b). Moreover, the distal leg portion 17a (17b) is connected for axial rotation about its longitudinal axis X3 to the proximal leg portion 16a (16b). Finally, the attachment device 9 is connected for rotation about a transverse axis X4 to the free end of the distal leg portion 17a (17b). All the angular displacements of the different leg portions and the attachment devices are carried out by tre relevant motors under external control, as will be explained below.

In the present embodiment the connection between the two “snake-like” robotic units 11 and 12 of each surgical robot 1 and 2 is carried out in such a way that the portions of proximal legs 16a and 16b of the unit 11 are coplanar to the corresponding portions of proximal legs 16a and 16b of the unit 12 as shown in FIG. 2.

It is clear from the foregoing that, once the surgical robot 1 or 2 is secured to the body cavity wall through the attachment legs 7a, b, c or 8a, b, c, the relative positions of the central bodies 10 of the surgical robots 1 and 2 can be varied in a wide range. This results in a far greater number of relative positions and orientations the surgical tools 5 and 6 are able to take on as compared to the prior art devices, such as those according to WO2007/149559 or WO2007/111571, in which the arms bearing the surgical tools extend from a common supporting means having a fixed spatial positioning. Furthermore, since the central bodies 10 are supported by three legs 7a, b, c and 8a, b, c respectively, the arms 3 and 4 bearing the surgical tools 5 and 6 can have better performances in term of force torque and reliability as compared to the prior art.

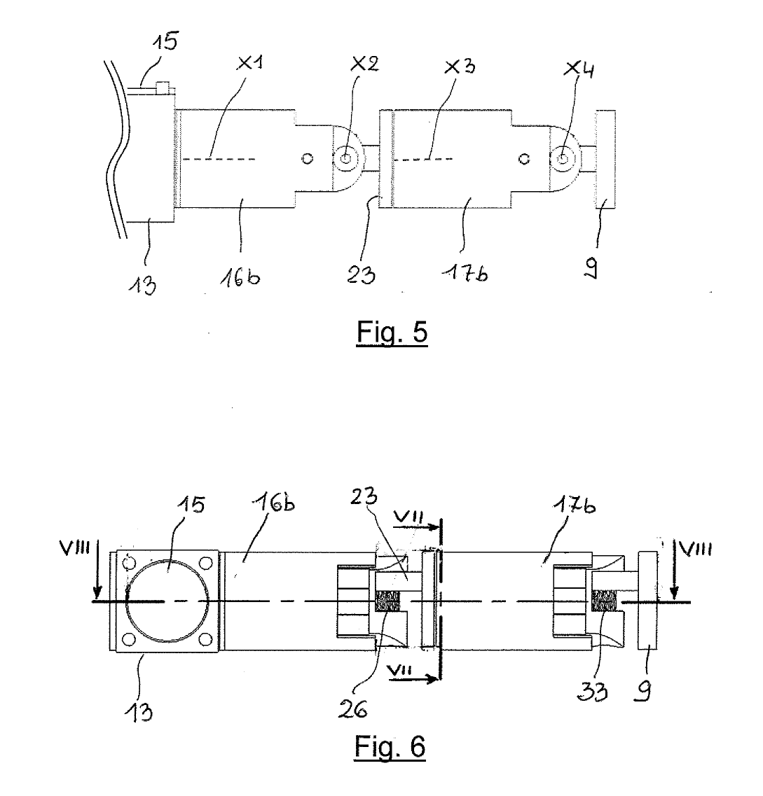

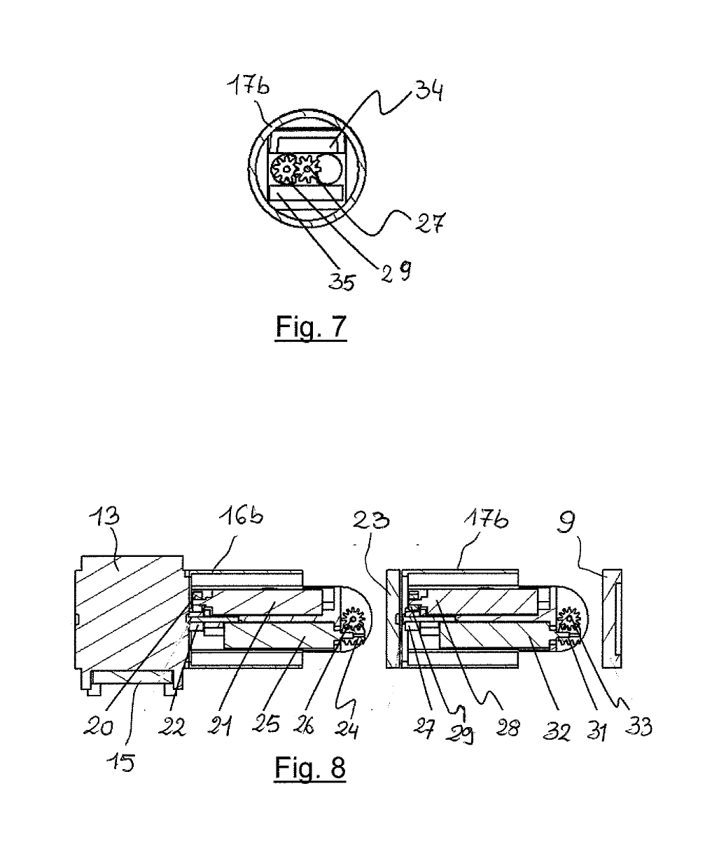

FIGS. 5 to 8 illustrate a possible constructional embodiment for an attachment leg of a surgical robot of the invention, the same technical solution applying to an operating arm 3 or 4. The proximal leg portion 16a is connected to the central unit 13 and rotates using a gear transmission formed by a spur gear 20 connected to a motor 21, housed in the proximal leg portion 16a, and engaging with a spur gear 22 integral to the central unit 13. The proximal leg portion 16a and the distal leg portion 17a are connected by a joint 23 allowing their relative bending about transverse axis X2. A worm 24 extends from a motor 25 housed in the proximal leg portion 16a and is engaged with a worm gear 26. The worm gear 26 is fixed to the joint 23, whereby relative bending is enabled once the motor 25 is started. The distal leg portion 17a is connected to the joint 23 and rotates using a gear transmission formed by a spur gear 27 connected to a motor 28, housed in the distal leg portion 17a, and engaging with a spur gear 29 integral to the joint 23. The distal leg portion 17a and the attachment device 9 are connected by a joint 30 allowing the attachment device 9 to rotate about transverse axis X4. A worm 31 extends from a motor 32 housed in the distal leg portion 17a and is engaged with a worm gear 33 fixed to the joint 30, whereby the attachment device 9 is enabled to rotate relative to the distal leg portion 17a.

The motors 21, 25, 28 and 32 can be DC brushless motor and can also be equipped with an encoder, in order to have closed loop control of the motion.

It is worth noting that the various proximal and distal leg portions, as well as the various proximal and distal operative arm portions, are structurally equal, i.e. the attachment legs and the operative arms have a modular structure. This greatly simplifies their production and assembling.

Each snake-like robotic unit is equipped with means for energy and data transmission and with a set of sensors to perceive the robot position in a tri-dimensional space and to monitor in real time its performance. On board battery 34 and a control board 35 can also by mounted on each leg portion.

A laser fibre can also be mounted on the operating arm 3 or 4 and the laser fibre can be passed through the insertion port.

One or more robotic cameras 36 are also inserted in the body cavity and attached to the body cavity wall in the same way as the attachment legs. In particular, a robotic camera 36 comprises an attachment device 9, an active (motorized) joint 37 with one or two degrees of freedom, a CMOS or CCD camera, a lens system, an illumination module and means for energy and data transmission.

The endoluminal micro-robotic platform according to the invention is used in the following way.

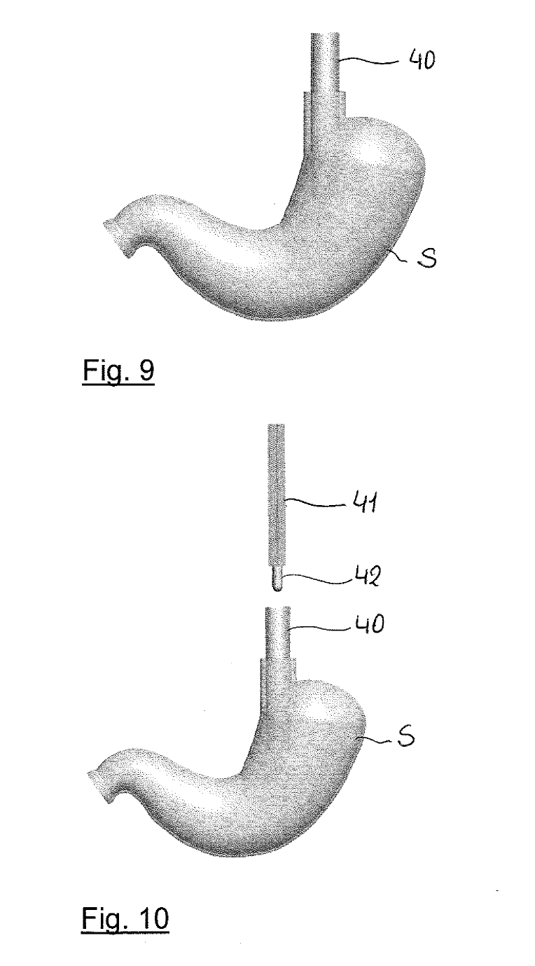

A semi-rigid gastro-esophageal insertion port 40 is introduced through the mouth into the gastric cavity of a sedated or anesthetized patient. The main functions of this port are to allow an easy and fast introduction of the different modules of the robotic platform and to maintain the stomach in an insufflated condition. See FIG. 9.

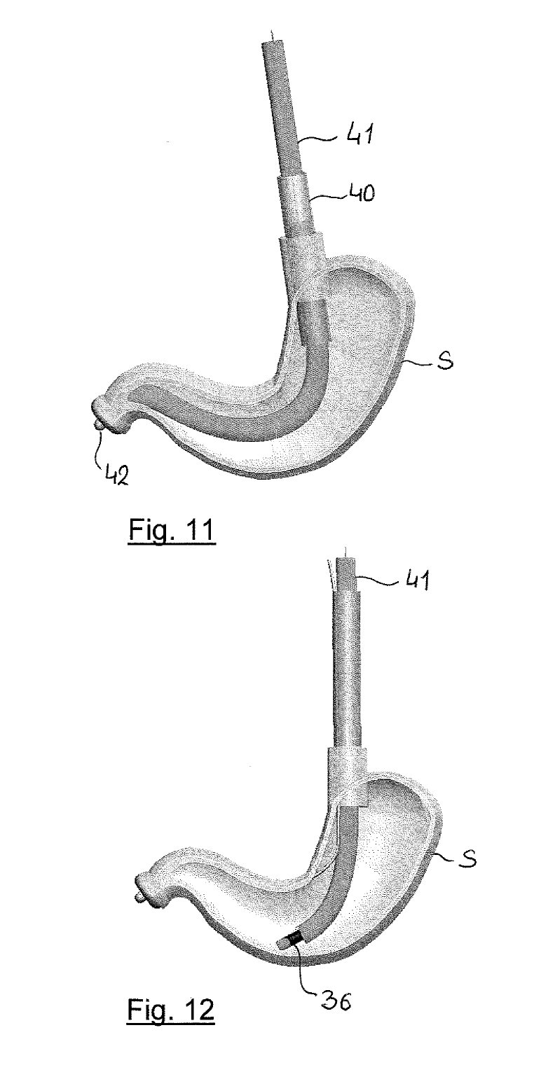

A flexible and externally steerable introducer 41 is used to deploy the different parts of the platform in the desired positions. To that end an auxiliary pipe to be inserted through a flexible endoscope can also be used. First a sealing element 42 to close the gastro-duodenal junction is introduced, thus allowing the required stable insufflation of the stomach. The sealing element 42 is basically an inflatable balloon, shown in FIG. 10, before placement and insufflation, and in FIG. 11, after placement and insufflation.

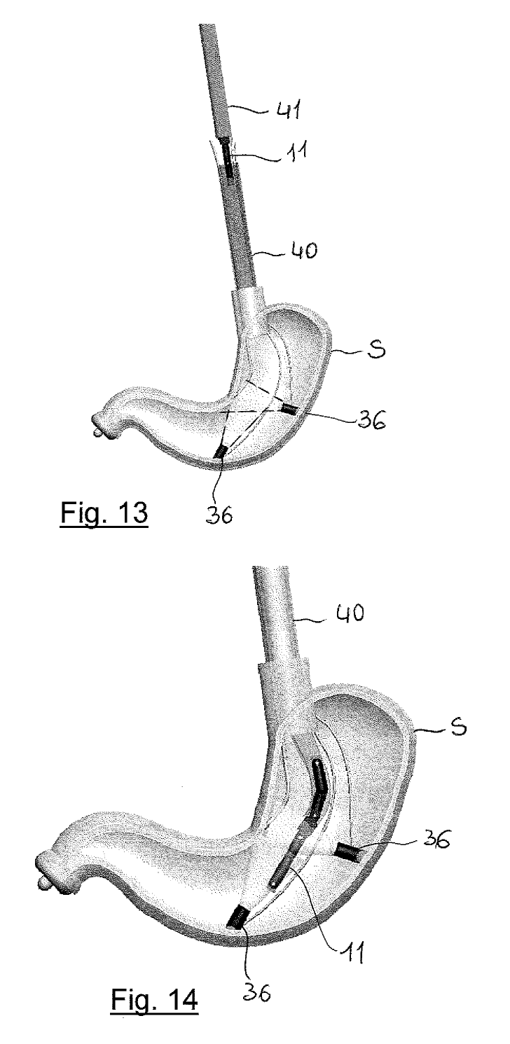

After the sealing element 42 has been placed, a set of deployable robotic cameras 36 is introduced, as shown in FIGS. 12 and 13. In the following the introduction of two robotic cameras in devised. In general the number of cameras depends on the specific surgical needs.

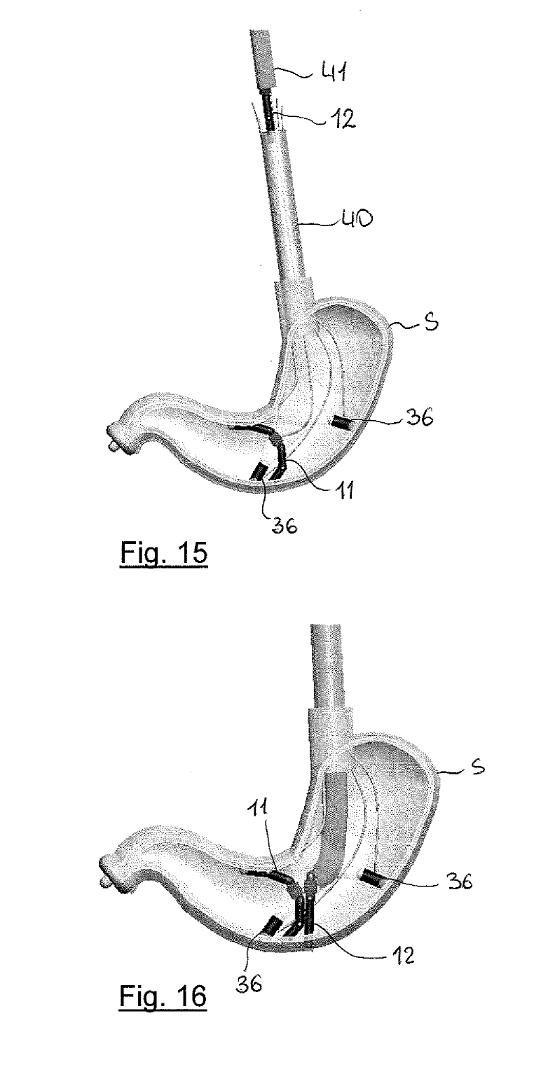

Then the first snake-like robotic unit 11 composing a first deployable surgical robot 1 is introduced, as shown in FIG. 13. The distal attachment device 9 is guided, under external control, to a first desired attachment position. Once the distal attachment device of the first snake-like robotic unit composing a first deployable surgical robot touches the gastric wall in the desired position, it sticks to the tissue, as shown in FIG. 14. Then the introducer can be withdrawn and the first snake-like robotic unit composing the first deployable surgical robot moves, under external control, in order to guide the other attachment device to a second desired attachment position on the gastric wall. When both the terminal attachment devices adhere to the gastric wall, as shown in FIG. 15, and the first snake-like robotic unit reaches a stable position.

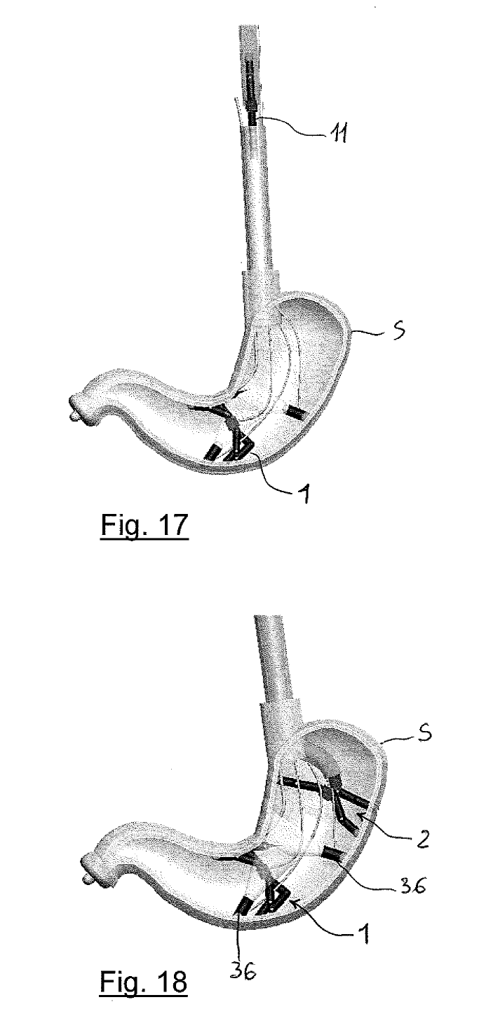

Once the first snake-like robotic unit composing the first deployable surgical robot holds a stable position, as shown in FIG. 15, the second snake-like robotic unit 12 composing a first deployable surgical robot 1 is introduced, as shown in FIG. 16, and positioned so that the central units of the two snake-like robotic units 11 and 12 can easily link together through the electro-mechanical linking mechanism. Once a proper connection of the two units is occurred, the introducer is withdrawn, and the attachment devices of the second snake-like robotic unit composing a first deployable surgical robot 1 is moved under external control towards the gastric wall, in order to achieve a stable adhesion. This procedure allows the correct assisted assembly of a first deployable surgical robot 1, having three legs, equipped with attachment devices at each free end, and an operating arm, equipped with a surgical tool, as shown in FIG. 17.

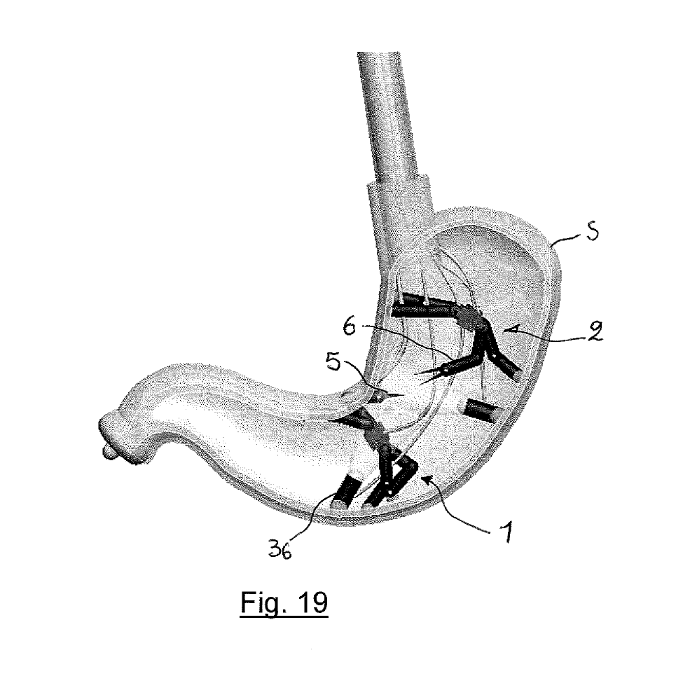

The same procedure, as shown in FIGS. 17 and 18, is followed to assemble a second deployable surgical robot 2, equipped with a different surgical tool, thus enabling an effective bimanual robotic surgery from inside the gastric cavity, as shown in FIG. 19.

Even if in the above description of the use of the endoluminal robotic platform according to the invention reference has been made to the gastric cavity as a body cavity, it is understood that the invention is not limited to this use and the endoluminal robotic platform of the invention can be deployed in any other body cavity through any other suitable natural or artificial orifice.

Various modifications and alterations to the invention may be made based on a review of the disclosure. These changes and addition are intended to be within the scope of the invention as set forth in the following claims.

1. An endoluminal micro-robotic platform deployable in a body cavity comprising:
a first and a second surgical robot each provided with a surgical tool and being configured to be attached to a body cavity wall of the body cavity, said first and second surgical robot each comprising a first and a second snake-like robotic unit;
the first snake-like robotic unit comprising a first central unit and a first articulated attachment means extending from said first central unit for attaching the first central unit to the body cavity wall;
the second snake-like robotic unit comprising a second central unit, a second articulated attachment means for attaching the second central unit to the body cavity wall and an articulated arm bearing said surgical tool, said second articulated attachment means and said articulated arm extending from said second central unit, a releasable connection means being provided on said first and second central units to connect the first central unit to the second central unit releasably to form each of said first and second surgical robot within said body cavity deployed in such a way to allow a surgical procedure to be performed in a true bimanual operation.

2. The endoluminal micro-robotic platform according to claim 1, wherein said first articulated attachment means comprise a pair of articulated attachment legs extending from opposite sides of said central unit and an attachment device at a free end of said legs.

3. The endoluminal micro-robotic platform according to claim 1, wherein said second articulated attachment means comprise an articulated attachment leg extending from one side of said central unit and an attachment device at a free end of said leg.

4. The endoluminal micro-robotic platform according to claim 2, wherein each articulated leg of said first and second articulated attachment means comprise at least two leg portions having longitudinal axes, a first one of said leg portions being pivotally connected to said central unit about its longitudinal axis and being connected to a second one of said leg portions for rotation about a transverse axis and the longitudinal axis of said second leg portion, said attachment device being pivotally connected to the free end of said leg.

5. The endoluminal micro-robotic platform according to claim 1, wherein said articulated operating arm bearing a surgical tool comprises at least two arm portions having longitudinal axes, a first one of said arm portions being pivotally connected to said central unit about its longitudinal axis and being connected to a second one of said arm portions for rotation about a transverse axis and the longitudinal axis of said second arm portion, said surgical tool being pivotally connected to the free end of said arm.

6. The endoluminal micro-robotic platform according to claim 1, wherein motor means are provided within said leg portions and arm portions to drive the rotational movements thereof under external control.

7. The endoluminal micro-robotic platform according to claim 1, wherein the attachment device comprises an adhesive polymer layer or an air suction device or a magnetic device.

8. The endoluminal micro-robotic platform according to claim 1, wherein said central unit comprises a local electronic control circuit and a means for wired or wireless energy and data transmission.

9. The endoluminal micro-robotic platform according to claim 1, further comprising at least one robotic camera comprising a body housing image sensing means, a lens system and illumination means, an attachment device for attaching the robotic camera to the body cavity wall, and a motorized joint for pivotally connecting the attachment device to said body.

10. A method for deploying an endoluminal micro-robotic platform in a body cavity for performing surgical procedures comprising:
providing at least two pairs of first and second snake-like robotic units according to claim 1 the previous claims,
introducing an insertion port through a natural orifice to the body cavity of a sedated or anesthetized patient,
introducing sealing elements to close any outlet port of said body cavity to keep the body cavity in an insufflated condition,
introducing at least one robotic camera to be attached to a selected point of the body cavity wall,
introducing a first snake-like robotic unit with two attachment legs to attach one leg of the attachment device to the body cavity wall in the desired position and then guiding, under external control, the other leg to attach the attachment device to another desired position of the body cavity wall,
introducing a second snake-like robotic unit with an attachment leg and an operating arm bearing a surgical tool to connect the central unit thereof to the central unit of the first snake-like robotic unit, thereby assembling a first surgical robot, and then guiding, under external control, the attachment device of the leg to attach it to the body cavity wall in the desired position, and
repeating the deploying operation for the second pair of first and second snake-like robotic units to assemble a second surgical robot in a desired position of the body cavity, thus deploying the endoluminal micro-robotic platform in the body cavity for performing surgical procedures.

11. The method according to claim 10, wherein the body cavity is the gastric cavity and the natural orifice is the mouth.

12. The method according to claim 10, wherein a flexible, externally steerable introducer is used to deploy the different components of the robotic platform in the desired positions.

13. The method according to claim 11, wherein a flexible, externally steerable introducer is used to deploy the different components of the robotic platform in the desired positions.

14. The endoluminal micro-robotic platform according to claim 3, wherein each articulated leg of said second articulated attachment means comprise at least two leg portions having longitudinal axes, a first one of said leg portions being pivotally connected to said central unit about its longitudinal axis and being connected to a second one of said leg portions for rotation about a transverse axis and the longitudinal axis of said second leg portion, said attachment device being pivotally connected to the free end of said leg.

15. The endoluminal micro-robotic platform according to claim 2, wherein motor means are provided within said leg portions to drive the rotational movements thereof under external control.

16. The endoluminal micro-robotic platform according to claim 3, wherein motor means are provided within said leg portions to drive the rotational movements thereof under external control.

17. The endoluminal micro-robotic platform according to claim 2, wherein the attachment device comprises an adhesive polymer layer or an air suction device or a magnetic device.

18. The endoluminal micro-robotic platform according to claim 3, wherein the attachment device comprises an adhesive polymer layer or an air suction device or a magnetic device.

19. The endoluminal micro-robotic platform according to claim 2, wherein said central unit comprises a local electronic control circuit and a means for wired or wireless energy and data transmission.

20. The endoluminal micro-robotic platform according to claim 3, wherein said central unit comprises a local electronic control circuit and a means for wired or wireless energy and data transmission.
